(19) United States
(12) Patent Application Publication
(10) Pub. No.: US 2019/0055724 A1
(43) Pub. Date: Feb. 21, 2019

(54) WATER-SOLUBLE AIR FRESHENER DELIVERY SYSTEM AND METHOD OF USING THE SAME

(71) Applicant: SOLUTIONS THAT STICK, INC., KETCHUM, ID (US)

(72) Inventor: Kim Leone Castellano, Ketchum, ID (US)

(21) Appl. No.: 15/679,995

(22) Filed: Aug. 17, 2017

(51) Int. Cl. E03D 9/00; A47K 17/00; A61L 9/04; A61L 9/05

(52) U.S. Cl. CPC E03D 9/007; A61L 9/05; A61L 9/044; A47K 17/003

(57) ABSTRACT

A fragrance sheet includes a film agent, a humectant, and a fragrance. The fragrance sheet is substantially water soluble. The fragrance sheet is included in an envelope having permanent seal on a top surface thereof. A resealable zipper closure is disposed below the permanent seal. The envelope is opened and one of the fragrance sheets is removed from the envelope. The removed fragrance sheet is disposed into a vessel of water. The fragrance sheet is dissolved within the water to release a plurality of fragrance particles. A surface of the water is coated with the plurality of fragrance particles.

### TECHNICAL FIELD

The present disclosure relates to air fresheners and, more specifically, to a water-soluble air freshener delivery system and method of using the same.

### DISCUSSION OF THE RELATED ART

Air fresheners are widely used to release pleasant smells for the purpose of hiding unpleasant odors or otherwise providing a more comfortable environment. While air fresheners are commonly used in homes, vehicles and restrooms, these air fresheners tend to be installed at fixed locations. As users may wish to carry air fresheners with themselves, for example, for the purpose of use while in a restroom, portable forms of air fresheners tend to rely upon liquids, aerosols, and gels, which can be difficult to carry, may pose a risk of accidental release, and may be prohibited from airport terminals.

### SUMMARY

A fragrance sheet includes a film agent, a humectant, and a fragrance. The fragrance sheet is substantially water soluble. The film agent may be Pullulan. The humectant may be glycerin. The fragrance may be water-soluble. The fragrance may be oil-based. The fragrance sheet may be a thin film. The fragrance sheet may have a first planar surface that is substantially smooth and a second planar surface, opposite to the first planar surface, that is substantially rough. The fragrance sheet may have a thickness of between 0.01 mm and 1.0 mm. The fragrance sheet may have a thickness of between 0.04 mm and 0.05 mm. The fragrance sheet may be substantially rectangular with a width of between 4 cm and 8 cm and a height between 4 cm and 8 cm.

A package of fragrance sheets includes an envelope having a permanent seal on a top surface thereof. A resealable zipper closure is disposed below the permanent seal. One or more substantially water-soluble fragrance sheets are disposed within the envelope. Each fragrance sheet includes a film agent, a humectant, and a fragrance.

The film agent may be Pullulan and the humectant may be glycerin. Each fragrance may be water-soluble or oil-based. Each fragrance sheet is a thin film having a thickness of between 0.01 mm and 1.0 mm. Each fragrance sheet may be substantially rectangular with a width of between 4 cm and 8 cm and a height between 4 cm and 8 cm.

A method for using a fragrance sheet includes opening an envelope having one or more substantially water-soluble fragrance sheets disposed therein. One of the fragrance sheets is removed from the envelope. The removed fragrance sheet is disposed into a vessel of water. The fragrance sheet is dissolved within the water to release a plurality of fragrance particles. A surface of the water is coated with the plurality of fragrance particles.

The vessel of water may be a toilet bowl, and the method may further include flushing the toilet bowl and coating an inner surface of the toilet bowl, above a water line thereof, with the plurality of fragrance particles. Each of the one or more substantially water-soluble fragrance sheets includes a film agent, a humectant, and a fragrance. The film agent may be Pullulan and the humectant may be glycerin. Each fragrance sheet may be a substantially rectangular thin film having a thickness of between 0.01 mm and 1.0 mm, a width of between 4 cm and 8 cm, and a height of between 4 cm and 8 cm.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a schematic diagram illustrating a single water-soluble air freshener sheet in accordance with exemplary embodiments of the present invention;

FIG. 2 is a schematic diagram illustrating an envelope for packaging one or more water-soluble air freshener sheets in accordance with exemplary embodiments of the present invention;

FIG. 3 is a schematic diagram illustrating delivery of the water-soluble air freshener in accordance with exemplary embodiments of the present invention;

FIG. 4 is a schematic diagram illustrating the dissolution of a water-soluble air freshener sheet in accordance with exemplary embodiments of the present invention; and

FIG. 5 is a flow chart illustrating an approach for delivering the water-soluble air freshener sheet in accordance with exemplary embodiments of the present invention.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention relate to an air freshener delivery system that uses thin sheets that are substantially water soluble. Each sheet is a solid film that contain, among other ingredients, a fragrance. When a sheet is placed in contact with water, for example, by dropping a sheet into a toilet bowel, the sheet substantially dissolves, releasing the fragrance. The fragrance coats the surface of the water and is released into the air within several seconds.

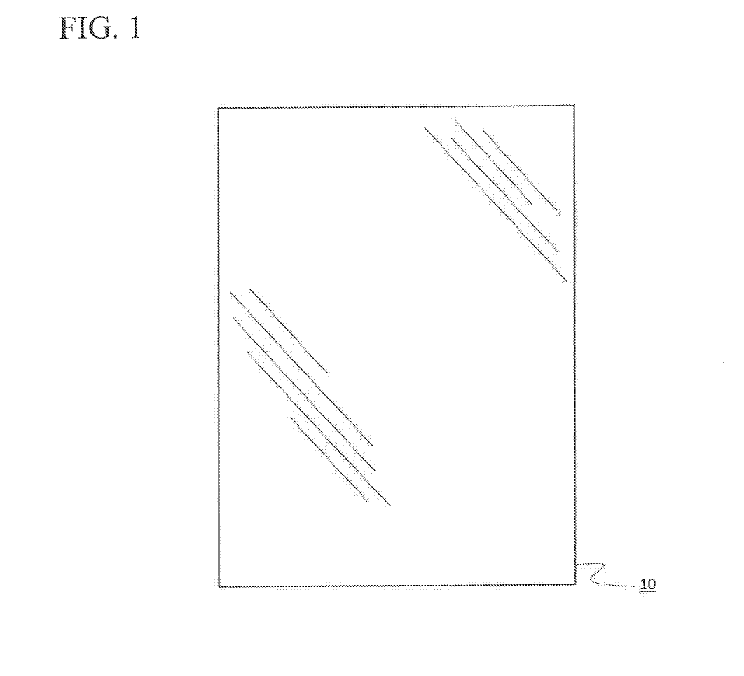

FIG. 1 is a schematic diagram illustrating a single water-soluble air freshener sheet in accordance with exemplary embodiments of the present invention. As can be seen, each sheet 10 may be substantially rectangular in shape. According to one exemplary configuration, the sheet 10 may be 6 cm in width and 8 cm in height. The thickness of the sheet may be between 0.01 and 1.0 mm, and may preferably be 0.05 mm (plus or minus 0.01 mm).

However, other shapes may be used. For example, each sheet 10 may be circular, oval, square, triangular, etc. Each sheet may be thin enough to dissolve quickly in water (for example, to enable significant dissolution within 1 to 30 seconds), but thick enough to deliver sufficient fragrance to mask unpleasant odors. While increasing the width and height (and thereby the surface area of the sheet), more fragrance may be delivered at a given thickness, exemplary embodiments of the present invention may use a sheet that is small enough in width and height to be conveniently carried. According to one exemplary approach, each sheet may be small enough to fit in a credit card slot in a wallet. As a standard credit card may be sized at around 5.5 cm×8.5 cm, a sheet size of 6 cm×8 cm may be well suited for use in a standard wallet. According to other exemplary embodiments of the present invention, each sheet may be stored in an envelope (either single-use or resealable) that itself can fit within a credit card slot (e.g. 5.5 cm×8.5 cm), and accordingly, each sheet may be smaller than the envelope (e.g. 4 cm by 7 cm). Where each sheet is a shape other than rectangle, the shape may be sized so as to fit within one of the aforementioned rectangular shapes.

The weight of each sheet, relative to its surface area, may be set such that the sheet may be suspended by the surface tension of the water that it is dropped into, without the sheet sinking into the water. In this way, as the sheet dissolves, the fragrance particles and other constituent ingredients may more easily create a film barrier over the surface of the water.

Each sheet 10 may include several constituent ingredients. One ingredient may form the delivery substrate. The delivery substrate is preferably a chemical that dissolves quickly in water and is a solid at room temperature. The delivery substrate may also be capable of absorbing a fragrance that is either water based or oil based. Starches, modified starches, and the like may serve as a delivery substrate.

Each sheet 10 may further include a film agent that can keep the other constituent ingredients together as a singular element until dissolved in water. An example of a suitable film agent is Pullulan, which is a polysaccharide polymer consisting of maltotriose units, also known as α-1,4-; α-1,6-glucan′. Pullulan may have the following chemical formula:

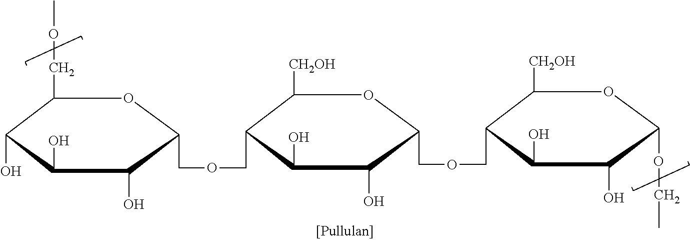

Other constituent ingredients may include a humectant such as glycerin, and a water-soluble and/or oil-based fragrance element. Water soluble fragrances may be used to quickly and evenly dissolve into water, while oil-based fragrance elements may be well suited to rise to the surface of the water and form a coating thereupon.

Other constituent ingredients may include menthol, which may have fragrance enhancing properties, and other useful functions.

According to one exemplary embodiment of the present invention, pullulan may constitute 60% to 80% (e.g. 68%) of the total ingredients, by weight; starch (e.g. corn starch/modified starch) may constitute 15% to 35% (e.g. 23%) of the total ingredients, by weight; menthol may constitute 1% to 10% (e.g. 4%) of the total ingredients, by weight; glycerin may constitute 1% to 10% (e.g. 5%) of the total ingredients, by weight. Fragrance ingredients may together constitute less than 1% of the total ingredients, by weight, however, according to other exemplary embodiments of the present invention, the fragrance may be increased, for example, to account for 1% to 10% (e.g. 5%) of the total ingredients, by weight.

Each sheet 10 may be substantially translucent and may be a cloudy white in appearance. This may be achieved, for example, by omitting dyes and other coloring agents from the composition of the sheet, as dyes and coloring agents might tend to leave color behind, which might not be desirable for use in public restrooms. Moreover, due to the water-soluble nature of the sheet, inclusion of dyes and coloring agents might tend to leave colored residue on the user's hands after handling the sheet.

The sheets may be formed to have a waxy but rough texture which may aid in removing the sheets from the package without the sheets sticking together. According to one exemplary embodiment of the present invention, each sheet may have a substantially smooth side and a substantially textured (e.g. rough) side. Then, the sheets may be stacked within its packaging such that the smooth side of one sheet is in contact with the textured side of the proximate sheet so that individual sheets may be easily removed and handled without sheets sticking to one another and to reduce the extent to which the sheet may leave residue on the hands of the user who handles it. The textured nature of the sheet may also serve to speed up the rate of dissolution in water.

While the sheets may be substantially rectangular in shape, and fully continuous, some exemplary embodiments of the present invention may have one or more cutouts, slits, holes, or other perforations in the sheet. These elements may speed up the rate of dissolving once placed in water.

The water-soluble air freshener sheets may be substantially water soluble, which herein means that the sheet itself will quickly break apart in water. It is noted, however, that one or more of the constituent ingredients need not be water soluble themselves, and as discussed above, one or more constituent ingredients may rise to the surface of the water and coat the surface of the water so that fragrance may be more effectively released and unpleasant odors may remain trapped below the surface of the water.

The sheets may be formed by one or more methods. For example, the ingredients may be mixed and heat cured. The heat-cured product may then be pressed, rolled, shaped and/or punched to the desired shape and thickness. The product may also be heat-cured and shaped into a block and then sheets may be shaved from the block. Finished sheets may be filled into packages.

Alternatively, the various ingredients may be spin-coated onto a surface and then peeled therefrom. According to another approach, the ingredients may be sprayed onto a surface, either in a mixed form or individually sprayed, and then the sheet may be peeled from the surface. Alternatively, an ink-jet process may be used to produce the sheets.

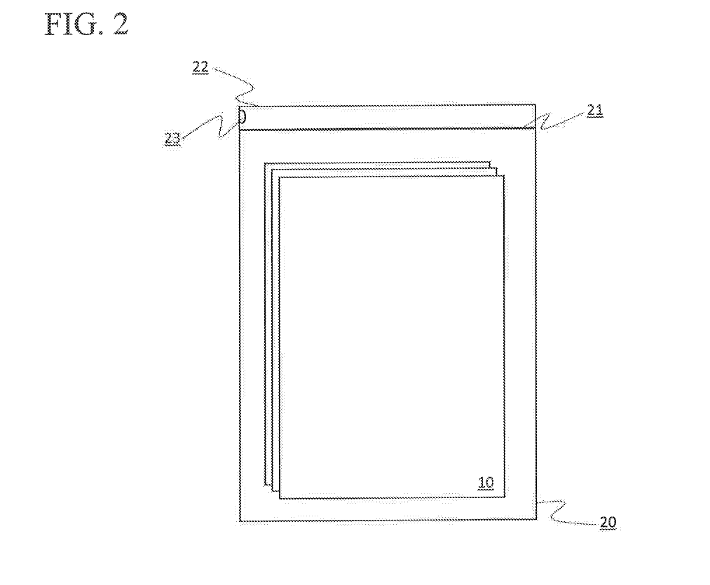

FIG. 2 is a schematic diagram illustrating an envelope for packaging one or more water-soluble air freshener sheets in accordance with exemplary embodiments of the present invention. As can be seen from this figure, the envelope package 20 may include multiple sheets 10. For example, the envelope package 20 may include twenty sheets 10. However, it is to be understood that the envelope package 20 may be designed to accommodate any number of sheets 10.

According to some exemplary embodiments of the present invention, the envelope package 20 may have a resealable opening 21. The resealable opening may be a zipper opening in which a structure of one side of the opening engages into a structure of an opposite side of the opening. The envelope package 20 may also include a secondary seal at the top of the envelope package 20 that is opened by a tearing away of the top potion 22 of the envelope package 20. One or more notches 23 may be included at the top of the envelope package 20 to facilitate the tearing away of the top portion 22.

According to another exemplary embodiment of the present invention, the envelope package 20 may include only a single sheet 10 and in this case, the envelope package 20 may be a single-use package. The single-use package need not include the resealable opening 21 and might only include the sealed top portion 22 and the one or more notches 23 for facilitating tearing.

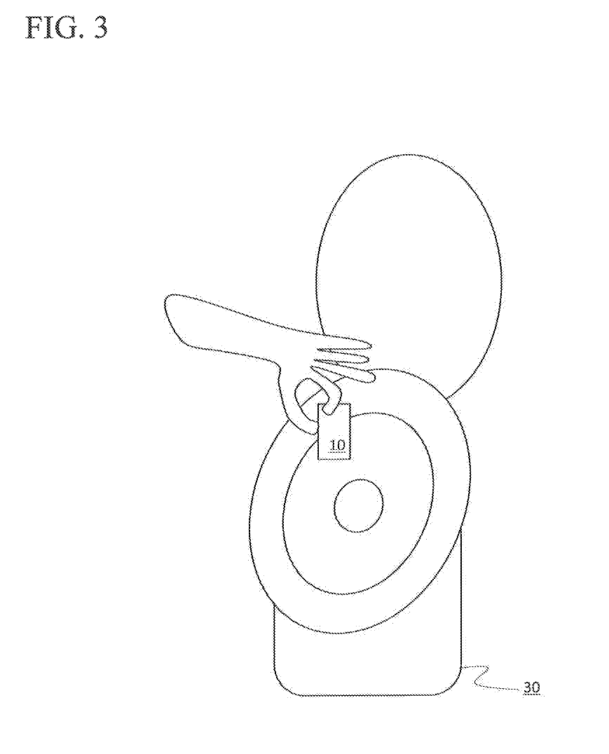

FIG. 3 is a schematic diagram illustrating delivery of the water-soluble air freshener in accordance with exemplary embodiments of the present, invention. The sheet 10 may be removed from the envelope package 20 and then dropped into a toilet bowel 30 or sink that contains water. The sheet 10 may be supported on the surface of the water by surface tension so that the sheet may dissolve in the water and the fragrance elements may coat the surface of the water.

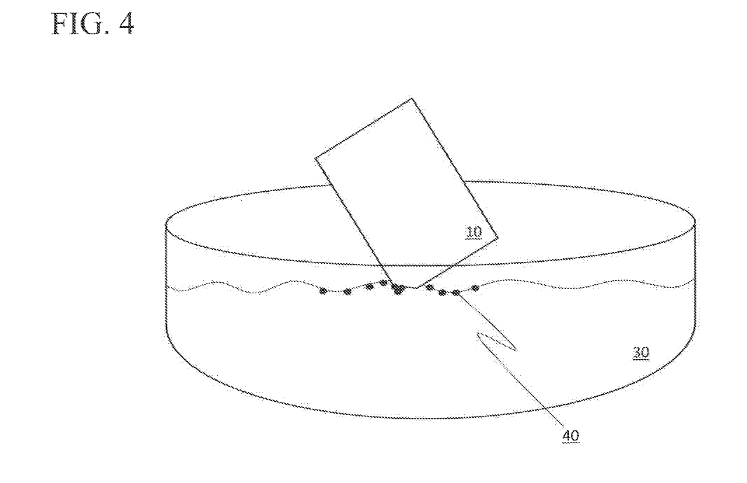

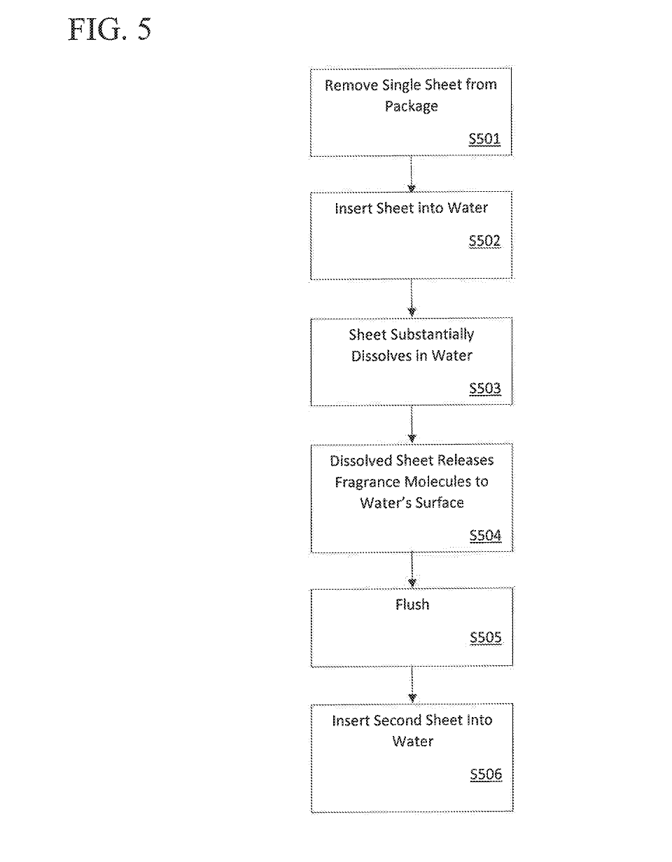

FIG. 4 is a schematic diagram illustrating the dissolution of a water-soluble air freshener sheet in accordance with exemplary embodiments of the present invention. FIG. 5 is a flow chart illustrating an approach for delivering the water-soluble air freshener sheet in accordance with exemplary embodiments of the present invention. With respect to FIGS. 4 and 5, the sheet 10 is removed from the package (Step S501) and then dropped or otherwise inserted into the water (Step S502). The sheet 10, having been dropped within the toilet bowl 30 or other vessel of water, may substantially dissolve (Step S503). As the sheet dissolves 10, fragrance particles 40 may be released into the water and may coat the surface of the water thereby forming a film over the surface of the water, for example, over the entire surface of the water (Step S504). The fragrance film may thereafter act to release a pleasant fragrance into the air and to suppress unpleasant odors from the toilet bowel.

Then, when the toilet is flushed (Step S505), the surface of the water is disrupted and additional fragrance is released into the air. Additionally, the disruption of the surface of the water may cause the fragrance film to line the surfaces of the toilet that are above the water line so that the release of fragrance into the air may continue even after flushing. However, a second sheet may be optionally dropped into the toilet after flushing for additional fragrance release (Step S506).

Exemplary embodiments described herein are illustrative, and many variations can be introduced without departing from the spirit of the disclosure or from the scope of the appended claims. For example, elements and/or features of different exemplary embodiments may be combined with each other and/or substituted for each other within the scope of this disclosure and appended claims.

1. A fragrance sheet, comprising:
a film agent;
a humectant; and
a fragrance,
wherein the fragrance sheet is substantially water soluble,
wherein the fragrance sheet has a first planar surface and a second planar surface, opposite to the first planar surface, and
wherein the first planar surface is smoother in texture than the second planar surface and the second planer surface is rougher in texture than the first planar surface.

2. The fragrance sheet of claim 1, wherein the film agent is Pullulan.

3. The fragrance sheet of claim 1, wherein the humectant is glycerin.

4. The fragrance sheet of claim 1, wherein the fragrance is water-soluble.

5. The fragrance sheet of claim 1, wherein the fragrance is oil-based.

6. The fragrance sheet of claim 1, wherein the fragrance sheet is a thin film.

7. The fragrance sheet of claim 1, wherein the fragrance sheet has a thickness of between 0.01 mm and 1.0 mm.

8. The fragrance sheet of claim 1, wherein the fragrance sheet has a thickness of between 0.04 mm and 0.05 mm.

9. The fragrance sheet of claim 1, wherein the fragrance sheet is substantially rectangular with a width of between 4 cm and 8 cm and a height between 4 cm and 8 cm.

10. (canceled)

11. A package of fragrance sheets, comprising:
an envelope having a permanent seal on a top surface thereof;
a resealable zipper closure disposed below the permanent seal; and
a plurality of substantially water-soluble fragrance sheets disposed within the envelope,
wherein each fragrance sheet of the plurality of fragrance sheets comprises:
a film agent;
a humectant; and
a fragrance,
wherein each fragrance sheet of the plurality of fragrance sheets has a first planar surface and a second planar surface, opposite to the first planar surface,
wherein the first planar surface is smoother in texture than the second planar surface and the second planar surface is rougher in texture than the first planar surface, and
wherein the plurality of fragrance sheets are disposed within the envelope such that a first planar surface of a first fragrance sheet of the plurality of fragrance sheets is in contact with a second planar surface of a second fragrance sheet.

12. The package of fragrance sheets of claim 11, wherein the film agent is Pullulan and the humectant is glycerin.

13. The package of fragrance sheets of claim 11, wherein each fragrance is water-soluble or oil-based.

14. The package of fragrance sheets of claim 11, wherein each fragrance sheet is a thin film having a thickness of between 0.01 mm and 1.0 mm.

15. The package of fragrance sheets of claim 11, wherein each fragrance sheet is substantially rectangular with a width of between 4 cm and 8 cm and a height between 4 cm and 8 cm.

16. A method for using a fragrance sheet, comprising:
opening an envelope having a plurality of substantially water-soluble fragrance sheets disposed therein;
removing one of the fragrance sheets from the envelope;
disposing the removed fragrance sheet into a vessel of water;
dissolving the fragrance sheet within the water to release a plurality of fragrance particles; and
coating a surface of the water with the plurality of fragrance particles,
wherein the plurality of fragrance sheets includes at least a first fragrance sheet and a second fragrance sheet,
wherein each of the plurality of fragrance sheets has a substantially smooth planar surface and a substantially rough planar surface, opposite to the substantially smooth planar surface,
wherein the substantially smooth planar surface of each of the plurality of fragrance sheets is smoother in texture than the substantially rough planar surface and the substantially rough planar surface is rougher in texture than the substantially smooth planar surface, and
wherein in removing one of the fragrance sheets from the envelope, the smooth side of the first fragrance sheet slides against the rough side of the second fragrance sheet or the rough side of the first fragrance sheet slides against the smooth side of the second fragrance sheet.

17. The method of claim 16, wherein the vessel of water is a toilet bowl, and the method further comprises flushing the toilet bowl and coating an inner surface of the toilet bowl, above a water line thereof, with the plurality of fragrance particles.

18. The method of claim 16, wherein each of the one or more substantially water-soluble fragrance sheets comprises:
a film agent;
a humectant; and
a fragrance.

19. The method of claim 18, wherein the film agent is Pullulan and the humectant is glycerin.

20. The method of claim 18, wherein each fragrance sheet is a substantially rectangular thin film having a thickness of between 0.01 mm and 1.0 mm, a width of between 4 cm and 8 cm, and a height of between 4 cm and 8 cm.

21. The package of fragrance sheets of claim 11, wherein the plurality of fragrance sheets are disposed within the envelope such that a first planar surface of the second fragrance sheet is in contact with a second planar surface of a third fragrance sheet.
